(19) United States
(12) Patent Application Publication
(10) Pub. No.: US 2026/0133110 A1
(43) Pub. Date: May 14, 2026

(54) IN VITRO TRANS-DERMAL DIFFUSION APPARATUS FOR GAS EXPOSURE

(71) Applicant: GUANGDONG UNIVERSITY OF TECHNOLOGY, Guangzhou (CN)

(72) Inventors: Yan YANG, Guangzhou (CN); Haojia CHEN, Guangzhou (CN); Zhiqin LIANG, Guangzhou (CN)

(21) Appl. No.: 19/024,005

(22) Filed: Jan. 16, 2025

(30) Foreign Application Priority Data
Sep. 13, 2024 (CN) 202411281235.2

(51) Int. Cl. G01N 13/00; G06T 7/00; G06T 7/11; G06T 7/62; G06T 17/00; G06T 19/20

(52) U.S. Cl. CPC G01N 13/00; G06T 7/0002; G06T 7/11; G06T 7/62; G06T 17/00; G06T 19/20; G01N 2013/003; G06T 2200/04; G06T 2219/2004

(57) ABSTRACT

An in vitro trans-dermal diffusion apparatus for gas exposure, including: a gas generator, an exposure chamber, an exhaust treating device, a sampling arm, a diffusion chamber, a liquid replenishment tank, and a sample bottle rack. The in vitro trans-dermal diffusion apparatus for gas exposure supports multiple diffusion chambers conducting gas exposure experiments simultaneously. The sampling port and replenishing port of the diffusion chamber are separated, enabling the rapid replenishment of fluids through a replenishment tank after sampling, which reduces the time the skin membrane is separated from the receptor fluid. The sampling arm of the in vitro trans-dermal diffusion apparatus for gas exposure, combined with a telescoping rod, allows the sampling needle to be inserted into the sampling port for sampling and can also be extended into a sample bottle to release the sample, minimizing the contact between the sample liquid and the air.

### CROSS REFERENCE TO RELATED APPLICATIONS

This application claims foreign priority of Chinese Patent Application No. 202411281235.2, filed on Sep. 13, 2024 in the China National Intellectual Property Administration, the disclosures of all of which are hereby incorporated by reference.

### TECHNICAL FIELD

The present disclosure relates to the technical field of trans-dermal experimental analysis and detection technology, in particular to an in vitro trans-dermal diffusion apparatus for gas exposure.

### BACKGROUND

Regarding the environmental pollution issues brought by rapid industrial development, especially the increasing emissions of volatile organic compounds (VOCs), the potential threats these substances pose to human health have gained widespread attention. Due to their high volatility and biological activity, volatile organic pollutants can penetrate the human body through skin contact, potentially leading to long-term toxic effects. However, accurately assessing the process and extent of percutaneous absorption of these gaseous pollutants has been a challenge in both scientific research and environmental protection fields. Current in vitro diffusion apparatus designs are mainly limited to measuring the percutaneous absorption of liquids, semi-solids, and solids, while there is a lack of corresponding equipment for measuring the percutaneous absorption of gases in the market, making it difficult to conduct accurate and effective in vitro experiments.

There are several key issues exist in existing technologies: (1) incomplete degassing leading to the gradual accumulation of bubbles, which reduces the permeation area and affects the accuracy of experimental results; (2) the use of the same pipeline for both sampling and liquid replenishment operations, increasing experimental errors; (3) the long time interval between automatic sampling and liquid replenishment in existing diffusion apparatus, causing prolonged separation between the skin membrane and receptor fluid, leading to inaccurate experimental results; (4) most current technologies only support a single set of gas exposure percutaneous experiments, and there is a lack of instruments that allow multiple diffusion cells to simultaneously perform gas exposure percutaneous experiments.

### SUMMARY

The present disclosure provides an in vitro trans-dermal diffusion apparatus for gas exposure, to solve the technical problem that existed in the prior arts.

To realize the above objective, the present disclosure provides an in vitro trans-dermal diffusion apparatus for gas exposure, including a gas generator, an exposure chamber, an exhaust treating device, a sampling arm, a diffusion chamber, a liquid replenishment tank, and a sample bottle rack; the diffusion chamber includes a skin cover, a receptor pool, gaskets, a skin membrane, a gas collecting port, a sampling port, and a liquid replenishment port; the skin membrane is clamped between the skin cover and the receptor pool, and each gasket is an elliptical ring; and a gas collecting port is upwardly inclined at the receptor pool, and the gas collecting port is at a position of 1 to 2 mm below a top of the receptor pool, with the sampling port and the replenishment port positioned opposite each other at a bottom of the diffusion chamber; and the gas generator is configured to generate a target gas with constant humidity, and the target gas is configured to continuously enter the exposure chamber through a peristaltic pump at a constant speed via a heating tube; and the exposure chamber is a temperature-controlled box with gas inlet holes and outlet holes on both sides, the gas inlet holes and the outlet holes are connected to exhaust hoses; and mounting holes of the diffusion chamber are evenly distributed at the bottom of the exposure chamber; and the replenishment tank is connected to the peristaltic pump to deliver liquid to the replenishment port; and the exposure chamber is equipped with an air inlet and an air outlet; and the exhaust treating device is located on a side of the outlet and communicated with the air outlet.

Furthermore, the sampling arm includes a supporting column, a sliding rail, a first sampling arm, a second sampling arm, and a sampling needle, wherein the sliding rail is positioned at a top of the supporting column, and the first sampling arm is configured to move along the sliding rail via an X-axis stepper motor.

Furthermore, the second sampling arm includes a rotating joint and a telescopic rod, with a sampling needle holder connected to an end of the telescopic rod.

Furthermore, a top structure of the receptor pool is inclined elliptical hollow with a protrusion at an upper end; a top structure of the skin cover is inclined elliptical hollow, and a bottom of the skin cover is defined with a groove that matches the protrusion of the receptor pool.

Furthermore, the sample bottle rack is configured to hold sample bottles and wash solution bottles.

Furthermore, the in vitro trans-dermal diffusion apparatus for gas exposure further includes a camera.

The present disclosure also provides a control method for an in vitro trans-dermal diffusion apparatus for gas exposure, applied to the in vitro trans-dermal diffusion apparatus for gas exposure, including the following steps: when the in vitro trans-dermal diffusion apparatus for gas exposure is on a working state, capturing a real-time posture image of a sampling arm at a preset time point through a camera, and performing a reduce redundancy process to the real-time posture image to obtain a reduced redundancy real-time posture image; and constructing a real-time posture three-dimensional (3D) model of the sampling arm at the preset time point based on the reduced redundancy real-time posture image, and obtaining a preset posture 3D model of the sampling arm at the preset time point; and calculating an overall overlap between the real-time posture 3D model of the sampling arm and the preset posture 3D model at a same preset time point through a model attribute map method; and comparing the overall overlap with a preset threshold; and if the overall overlap between the real-time posture 3D model and the preset posture 3D model is greater than the preset threshold, which indicates that the posture of the sampling arm is normal, and no correction is applied to the sampling arm; and if the overall overlap is not greater than the preset threshold, which indicates that the posture of the sampling arm is abnormal posture, and an optimal correction parameter is generated to correct the sampling arm based on the optimal correction parameter.

Furthermore, the step of calculating an overall overlap between the real-time posture 3D model of the sampling arm and the preset posture 3D model at a same preset time point through a model attribute map method includes the following steps: constructing a virtual space, and importing the real-time posture 3D model and the preset posture 3D model into the virtual space, to obtain a real-time posture assembly reference plane of the real-time posture 3D model and a preset posture assembly reference plane of the preset posture 3D model; and in the virtual space, aligning the real-time posture assembly reference plane and the preset posture assembly reference plane, and merging the real-time posture assembly reference plane and the preset posture assembly reference plane to obtain a merged posture 3D model; and dividing the merged posture 3D model into several sub-regions, and each sub-region represents as a polygon; and for each sub-region, calculating a ratio of the overlap volume between the real-time posture 3D model and the preset posture 3D model to a total volume of the sub-region through Boolean operations, to obtain an overlap degree between the real-time posture 3D model and the preset posture 3D model in each sub-region; and aggregating overlap degrees of the sub-regions through a weighted sum method to calculate the overall overlap between the real-time posture 3D model and the preset posture 3D model.

Furthermore, the step of if the overall overlap is not greater than the preset threshold, which indicates that the posture of the sampling arm is abnormal posture, and an optimal correction parameter is generated to correct the sampling arm based on the optimal correction parameter includes the following steps: obtaining operation log information of the in vitro trans-dermal diffusion apparatus for gas exposure, and extracting abnormal posture 3D models corresponding to various abnormal postures of the sampling arm from the operation log; and if the overall overlap between the real-time posture 3D model and the preset posture 3D model is not greater than the preset threshold, obtaining the real-time posture 3D model of the sampling arm is obtained, and calculating the overall overlap between the real-time posture 3D model and the abnormal posture 3D models corresponding to abnormal postures of the sampling arm; selecting the abnormal posture 3D model corresponding to the maximum overall overlap, and retrieving historical correction parameters corresponding to the maximum overall overlap from the operation log information, and obtaining correction accuracy of the sampling arm after applying various historical correction parameters; constructing a size ranking table, and inputting the correction accuracy of the sampling arm after applying various historical correction parameters into the size ranking table for sorting, to obtain maximum correction accuracy; obtaining the historical correction parameters corresponding to the maximum correction accuracy, and delivering historical correction parameters corresponding to the maximum correction accuracy to a controller of the in vitro trans-dermal diffusion apparatus for gas exposure as the optimal correction parameter, and the controller is configured to correct the sampling arm based on the optimal correction parameter.

The present disclosure addresses the technical deficiencies present in the background technology and offers the following beneficial effects: the in vitro trans-dermal diffusion apparatus for gas exposure combines an exposure chamber with an in vitro transdermal diffusion instrument, which can be applied to gas exposure. Traditional gas exposure experiments are conducted using live animals; however, the in vitro trans-dermal diffusion apparatus for gas exposure extends the scope of gas exposure experiments to in vitro settings. Additionally, the in vitro trans-dermal diffusion apparatus for gas exposure replaces the donor chamber of a traditional diffusion chamber with a skin cover structure that can hold the skin, allowing the gas to fully contact the skin membrane and simulate a real skin surface environment. The in vitro trans-dermal diffusion apparatus for gas exposure supports multiple diffusion chambers conducting gas exposure experiments simultaneously. The sampling port and replenishing port of the diffusion chamber are separated, enabling the rapid replenishment of fluids through a replenishment tank after sampling, which reduces the time the skin membrane is separated from the receptor fluid. The sampling arm of the in vitro trans-dermal diffusion apparatus for gas exposure, combined with a telescoping rod, allows the sampling needle to be inserted into the sampling port for sampling and can also be extended into a sample bottle to release the sample, minimizing the contact between the sample liquid and the air.

### BRIEF DESCRIPTION OF THE DRAWINGS

In order to more clearly illustrate the technical solutions in the embodiments of the present disclosure or the prior art, a brief introduction to the drawings used in the descriptions of the embodiments or prior art is provided below. It is evident that the drawings described below are merely some embodiments of the present disclosure. For those skilled in the art, without making any effective efforts, other drawings of different embodiments can be derived from these drawings.

FIG. 1 is a schematic structural diagram of an in vitro trans-dermal diffusion apparatus for gas exposure according to an embodiment of the present disclosure.

FIG. 2 is a schematic structural diagram of a sampling arm of the in vitro trans-dermal diffusion apparatus according to an embodiment of the present disclosure.

FIG. 3 is a schematic structural diagram of a diffusion chamber of the in vitro trans-dermal diffusion apparatus according to an embodiment of the present disclosure.

FIG. 4 is an exploded diagram of the diffusion chamber in FIG. 3.

FIG. 5 is a schematic structural diagram of an exposure chamber of the in vitro trans-dermal diffusion apparatus according to an embodiment of the present disclosure.

FIG. 6 is a section view of a skin cover of the in vitro trans-dermal diffusion apparatus for gas exposure according to an embodiment of the present disclosure.

FIG. 7 is a flowchart of a control method for a in vitro trans-dermal diffusion apparatus for gas exposure according to an embodiment of the present disclosure.

FIG. 8 is a detailed flowchart of a control method for the in vitro trans-dermal diffusion apparatus for gas exposure according to an embodiment of the present disclosure.

FIG. 9 is another detailed flowchart of a control method for the in vitro trans-dermal diffusion apparatus for gas exposure according to an embodiment of the present disclosure.

FIG. 10 is a diagram of a control equipment for the in vitro trans-dermal diffusion apparatus for gas exposure.

### DESCRIPTION OF THE REFERENCE NUMERAL

1 supporting column, 2 sample bottle rack, 3 sampling needle holder, 4 exhaust treating device, 5 gas generator, 6 peristaltic pump, 7 liquid replenishment tank, 8 diffusion chamber, 9 exposure chamber, 10 second sampling arm, 11 first sampling arm, 12 sliding rail, 13 skin cover, 14 gasket, 15 skin membrane, 16 receptor pool, 17 gas collecting port, 18 sampling port, 19 liquid replenishment port, 20 buckle, 21 air inlet, 22 air outlet, 23 mounting hole, 24 diverter valve, 25 slot, 26 sampling needle, 101 rotating joint, 102 telescopic rod.

### DETAILED DESCRIPTION OF THE EMBODIMENTS

In order to better understand the aforementioned objectives, features, and advantages of the present disclosure, a more detailed description of the disclosure is provided below, in conjunction with the accompanying drawings and specific embodiments. These drawings are simplified schematic diagrams, which illustrate the basic structure of the present disclosure in a schematic manner and only display the relevant components related to the disclosure. It should be noted that, when there is no conflict, the embodiments and features within the embodiments in this application can be combined with each other.

In the description of this application, it should be understood that the terms “center”, “longitudinal”, “lateral”, “up”, “down”, “front”, “back”, “left”, “right”, “vertical”, “horizontal”, “top”, “bottom”, “inside”, “outside”, and other directional or positional relationships are based on the orientations or positional relationships shown in the accompanying drawings. These terms are merely for the convenience of describing the application and simplifying the description, and do not indicate or imply that the device or component must have a specific orientation or be constructed and operated in a specific orientation. Therefore, they should not be interpreted as limitations on the scope of protection of this application. In addition, the terms “first”, “second”, and so on are used only for descriptive purposes and should not be interpreted as indicating or implying relative importance or the number of technical features indicated. Therefore, features described with “first”, “second”, and similar terms may explicitly or implicitly include one or more of those features. In the description of the present disclosure, unless otherwise specified, the term “plurality” means two or more.

In the description of this disclosure, it should be noted that, unless explicitly defined otherwise, terms such as “install”, “connected”, and “coupled” should be broadly understood. For example, they could refer to a fixed connection, a detachable connection, or an integrated connection; a mechanical connection or an electrical connection; a direct connection or an indirect connection through an intermediary; or communication within two components. For those skilled in the art, the specific meaning of these terms in this application can be understood based on the specific circumstances.

For ease of understanding, the present disclosure will be described more comprehensively below with reference to the relevant drawings. The drawings provide an optimal embodiment of the present disclosure. However, the disclosure can be implemented in many different forms and is not limited to the embodiments described herein. On the contrary, the purpose of providing these embodiments is to facilitate a more thorough and comprehensive understanding of the disclosure of the present disclosure.

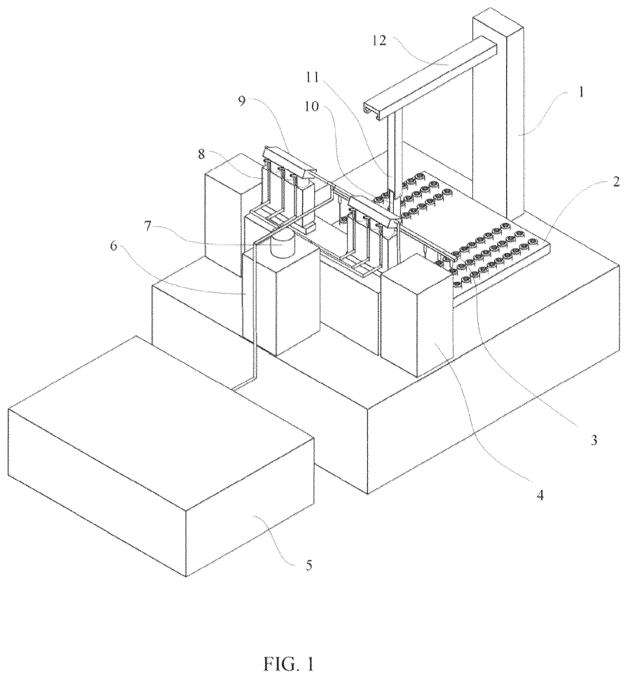

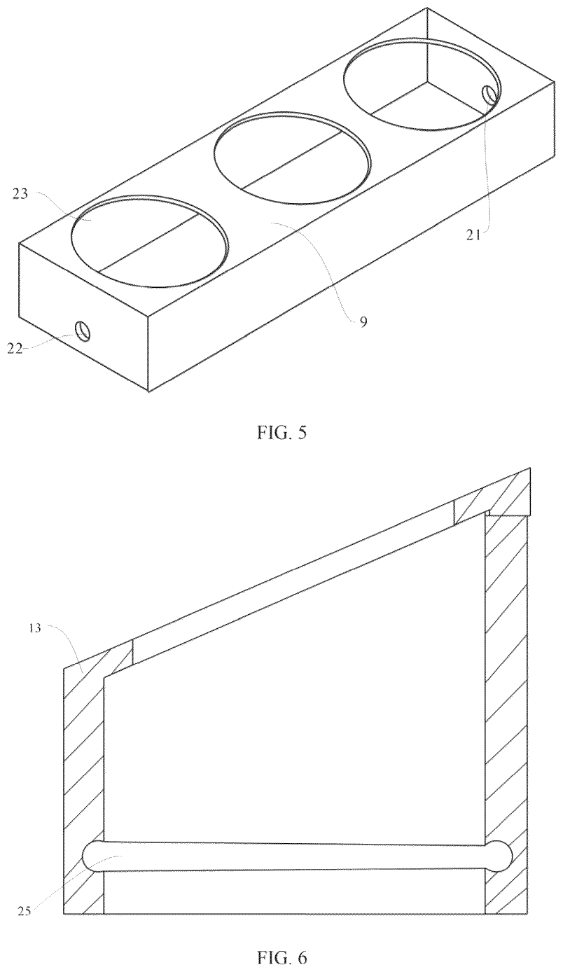

As shown in FIGS. 1 to 6, the present embodiment provides an in vitro trans-dermal diffusion apparatus for gas exposure.

As shown in FIG. 1, the in vitro trans-dermal diffusion apparatus for gas exposure includes a supporting column 1, a sample bottle rack 2, a sampling needle holder 3, an exhaust treating device 4, a gas generator 5, a peristaltic pump 6, a liquid replenishment tank 7, a diffusion chamber 8, an exposure chamber 9, a second sampling arm 10, a first sampling arm 11 and a sliding rail 12.

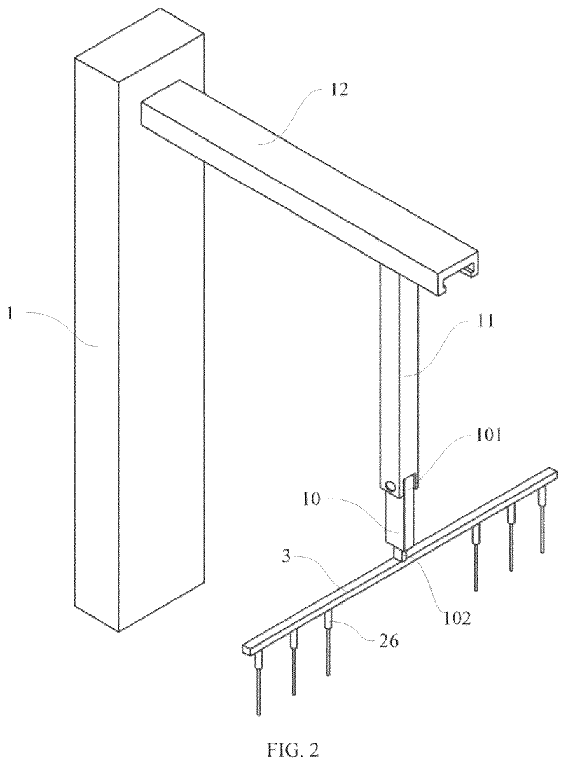

As shown in FIG. 2, a bottom of the sampling arm is mounted with a supporting column 1, on which a sliding rail 12 is installed. The first sampling arm 11 moves along an X-axis direction on the sliding rail 12. The second sampling arm 10 is consisted of a rotating joint 101 and a telescopic rod 102. The rotating joint 101 drives the telescopic rod 102 to rotate in a plane to position the sampling port 18, wash liquid bottles and sample bottles. The sampling needle holder 3 is located at a far end of the second sampling arm 10. After the rotating joint 101 is positioned, the telescopic rod 102 drives the sampling needle 26 into the sampling port 18 for sampling. Positioning and sampling operations are controlled through input signals from a controller. And the controller may be a PLC logic controller.

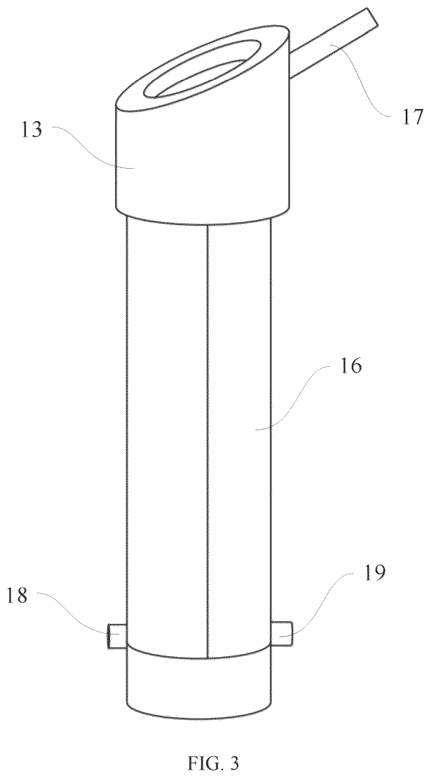

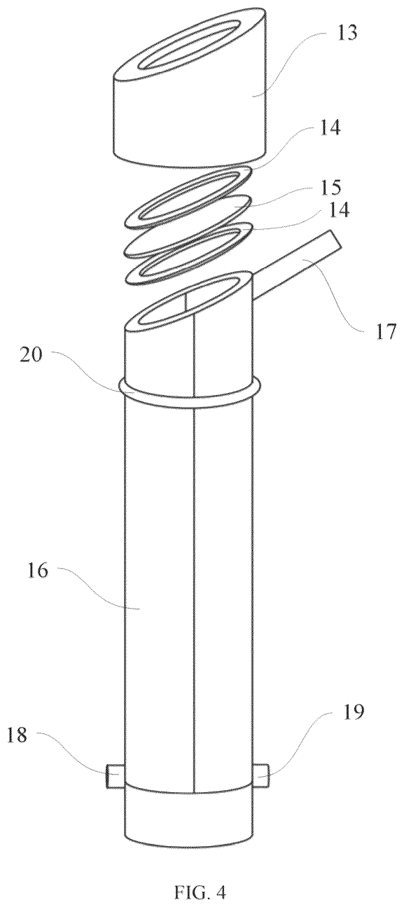

As shown in FIGS. 3 and 4, the diffusion chamber 8 includes a skin cover 13, a receptor pool 16, gaskets 14, a skin membrane 15, a gas collecting port 17, a sampling port 18, and a replenishment port 19. The top of the receptor pool 16 is inclined to fully guide the bubbles adhered to the skin membrane 15 into the gas collecting port 17. The skin cover 13 fits the contour of the receptor pool 16, and by applying pressure, the slot 25 of the skin cover 13 engages with the buckle of the receptor pool 16 to clamp the skin membrane 15. The gas collecting port 17 is inclined upward 1 to 2 mm below a top of the receptor pool 16.

As shown in FIGS. 1 and 5, the pollutants are evenly dispersed into the air by the gas generator 5, and generating target gas with a constant temperature and humidity. The target gas is split by a diverter valve 24 into two equal amounts and directed into the exposure chamber 9, where the skin membrane 15 is exposed to the target gas. The target gas slowly enters the skin membrane 15 and dissolves into the receptor liquid. The residual gas is absorbed by the exhaust treating device 4, which includes an activated carbon tower or pollutant absorption liquid bottle.

When applied in practice, within the sampling and replenishment system, after the sampling needle 26 is inserted into the sampling port 18, the PLC logic controller controls the stepper motor to pull the sampling piston forward to control the sampling volume. Once sampling is complete, the telescopic rod 102 retracts, and the replenishment tank 7 releases an equal amount of replenishment liquid into the receptor pool 16 via the peristaltic pump 6.

In the gas exposure system, when multiple diffusion chambers 8 are filled and installed in the exposure chamber 9, the target gas is introduced into the exposure chamber 9 and to begin the gas exposure experiment. During sampling, the sliding rail 12 carries the first sampling arm 11 to move above the diffusion chamber 8 that is ready for sampling. The rotating joint 101 rotates at a certain angle, and the second sampling arm 10 transitions from standby to sampling mode. The telescopic rod 102, mounted at an end of the second sampling arm 10, drives the sampling needle 26 into the sampling port 18 of the diffusion chamber 8 to complete the sampling. The sample is then moved by the stepper motor to the sample bottle rack 2 above the sample bottle. The rotating joint 101 rotates to align the sampling needle 26 with the sample bottle. The telescopic rod 102 drives the sampling needle 26 to extend into the sample bottle and release the sample, after which the needle is moved to the wash liquid bottle to clean the sampling needle 26, thus completing one sampling cycle.

It should be noted that the sampling and replenishment operation includes: programming set time points to control a movement of the sampling arm; positioning the sampling needle 26 by the rotating joint 101; driving the telescopic rod 102 to position the sampling needle for sampling at the sampling port 18; replenishing the liquid; extending the needle into the sample bottle to release the sample; and positioning it to the wash liquid bottle to clean the pipeline.

An upper part of the skin membrane in the diffusion chamber 8 is installed in the exposure chamber 9, while a lower part of the skin membrane is installed in a dry heating jacket. The contour of the dry heating jacket is compatible with the diffusion chamber 8. The temperature inside the exposure chamber 9 is controlled by the target gas, which regulates the temperature of a donor chamber to maintain a constant surface temperature of the skin membrane. The dry heating device keeps the receptor liquid in the receptor pool at a constant temperature of 37° C., simulating the temperature difference between the external and internal temperatures of normal human skin. The entire diffusion chamber's stirring system is driven by a stir bar, with a magnetic control device located directly beneath the chamber. The magnetic control device controls the stir bar's rotation speed, ensuring the receptor liquid is stirred constantly.

The in vitro trans-dermal diffusion apparatus for gas exposure integrates an exposure chamber with an in vitro trans-dermal diffusion instrument, making it suitable for gas exposure. Traditional gas exposure experiments use live animals, but the in vitro trans-dermal diffusion apparatus for gas exposure extends the range of gas exposure experiments to in vitro settings. Furthermore, the in vitro trans-dermal diffusion apparatus for gas exposure replaces the donor chamber in traditional diffusion chambers with a skin cover structure that can clamp the skin, allowing the gas to fully contact the skin membrane and simulate a real skin surface environment. It supports multiple sets of diffusion chambers to conduct gas exposure experiments simultaneously. The sampling port and replenishment port of the diffusion chamber are separated, allowing for the quick replenishment of liquid via the replenishment tank after sampling, reducing the time the skin membrane is separated from the receptor liquid. The sampling arm, combined with the telescopic rod, enables the sampling needle to enter the sampling port for sampling and also to extend into the sample bottle to release the sample, minimizing the contact between the sample liquid and air.

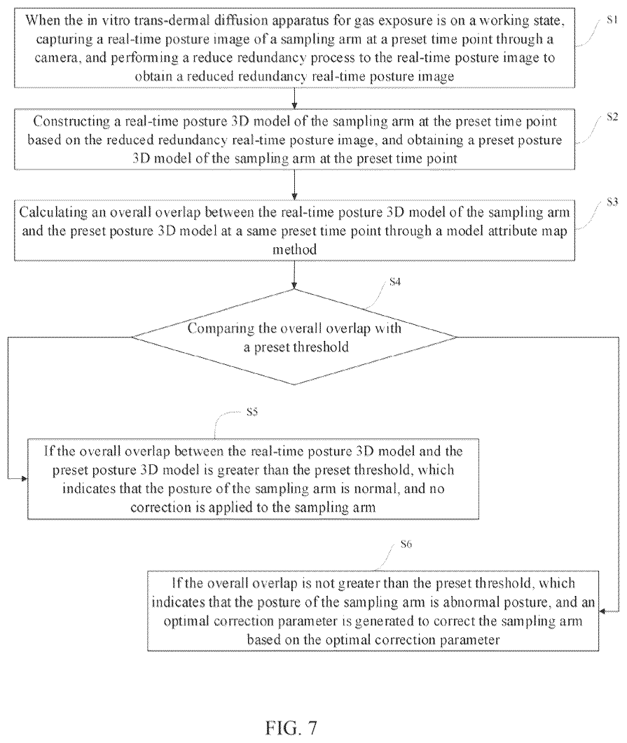

Another embodiment of the present embodiment disclosed a control method for an in vitro trans-dermal diffusion apparatus for gas exposure, applied to the in vitro trans-dermal diffusion apparatus for gas exposure mentioned above, as shown in FIG. 7, the method includes the following steps.

S1, when the in vitro trans-dermal diffusion apparatus for gas exposure is on a working state, capturing a real-time posture image of a sampling arm at a preset time point through a camera, and performing a reduce redundancy process to the real-time posture image to obtain a reduced redundancy real-time posture image.

S2, constructing a real-time posture three-dimensional (3D) model of the sampling arm at the preset time point based on the reduced redundancy real-time posture image, and obtaining a preset posture 3D model of the sampling arm at the preset time point.

S3, calculating an overall overlap between the real-time posture 3D model of the sampling arm and the preset posture 3D model at a same preset time point through a model attribute map method.

S4, comparing the overall overlap with a preset threshold.

S5, if the overall overlap between the real-time posture 3D model and the preset posture 3D model is greater than the preset threshold, which indicates that the posture of the sampling arm is normal, and no correction is applied to the sampling arm.

S6, if the overall overlap is not greater than the preset threshold, which indicates that the posture of the sampling arm is abnormal posture, and an optimal correction parameter is generated to correct the sampling arm based on the optimal correction parameter.

It should be noted that, through the method of real-time monitoring and correction of the sampling arm, it ensures that the sampling arm remains at the preset position throughout the working process. This allows the sampling needle to accurately align with the sampling port, sample bottle, and wash liquid bottle, thereby avoiding collisions, effectively improving the reliability of the device, while also preventing the occurrence of “mis-insertion” of the sampling needle, thus significantly improving the accuracy of the experiment. It should also be noted that the overall overlap degree calculated is output as a final result. The overall overlap degree is a value between 0 and 1, where 0 indicates complete non-overlap, and 1 indicates complete overlap. Based on the overlap degree, the similarity and differences between the two models are analyzed.

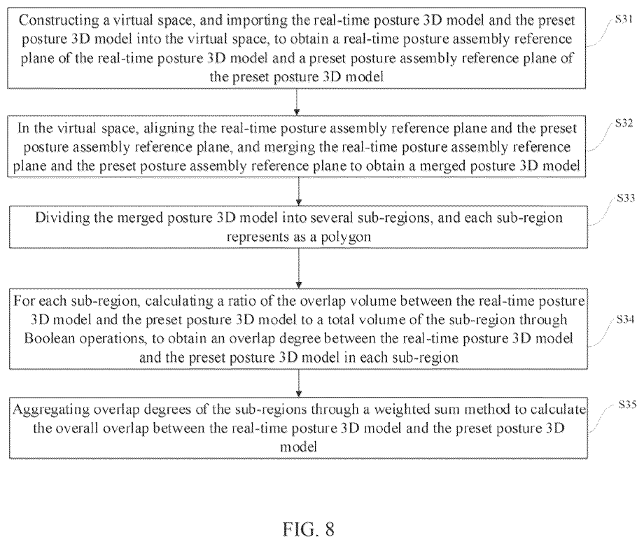

As shown in FIG. 8, the S3 of calculating an overall overlap between the real-time posture 3D model of the sampling arm and the preset posture 3D model at a same preset time point through a model attribute map method includes the following steps.

S31, constructing a virtual space, and importing the real-time posture 3D model and the preset posture 3D model into the virtual space, to obtain a real-time posture assembly reference plane of the real-time posture 3D model and a preset posture assembly reference plane of the preset posture 3D model; and

S32, in the virtual space, aligning the real-time posture assembly reference plane and the preset posture assembly reference plane, and merging the real-time posture assembly reference plane and the preset posture assembly reference plane to obtain a merged posture 3D model.

S33, dividing the merged posture 3D model into several sub-regions, and each sub-region represents as a polygon.

S34, for each sub-region, calculating a ratio of the overlap volume between the real-time posture 3D model and the preset posture 3D model to a total volume of the sub-region through Boolean operations, to obtain an overlap degree between the real-time posture 3D model and the preset posture 3D model in each sub-region.

S35, aggregating overlap degrees of the sub-regions through a weighted sum method to calculate the overall overlap between the real-time posture 3D model and the preset posture 3D model.

It should be explained that the operational log information of the in vitro trans-dermal diffusion apparatus for gas exposure refers to a series of data and information automatically recorded by the system during the experiment. These records include details about the operation status of the apparatus, operational processes, experimental parameters, and any abnormal conditions. This information is crucial for monitoring the test process, analyzing test results, optimizing experimental methods, and improving equipment design.

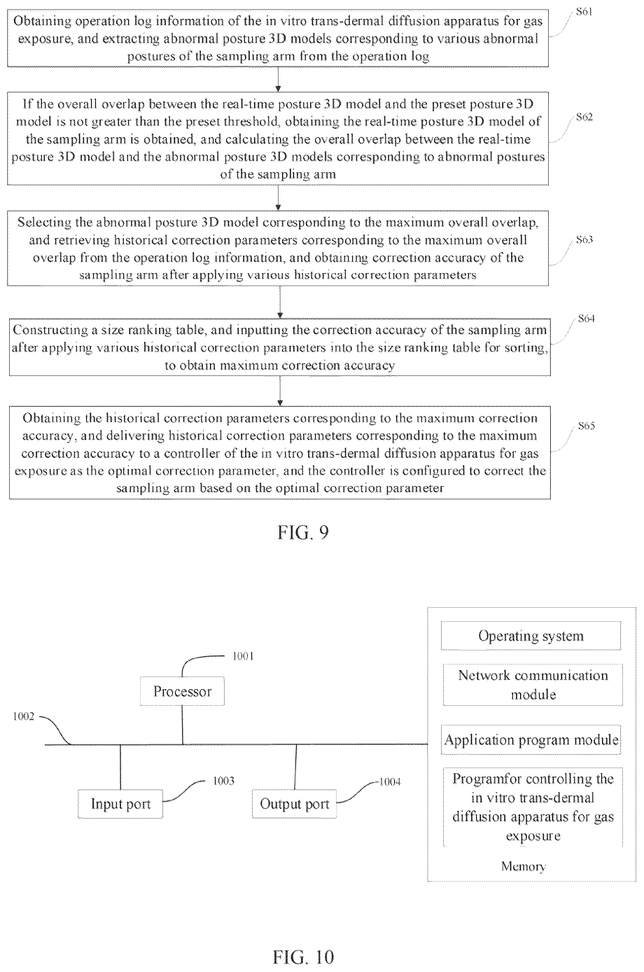

As shown in FIG. 9, the S6 of if the overall overlap is not greater than the preset threshold, which indicates that the posture of the sampling arm is abnormal posture, and an optimal correction parameter is generated to correct the sampling arm based on the optimal correction parameter including the following steps.

S61, obtaining operation log information of the in vitro trans-dermal diffusion apparatus for gas exposure, and extracting abnormal posture 3D models corresponding to various abnormal postures of the sampling arm from the operation log; and

S62, if the overall overlap between the real-time posture 3D model and the preset posture 3D model is not greater than the preset threshold, obtaining the real-time posture 3D model of the sampling arm is obtained, and calculating the overall overlap between the real-time posture 3D model and the abnormal posture 3D models corresponding to abnormal postures of the sampling arm;

S63, selecting the abnormal posture 3D model corresponding to the maximum overall overlap, and retrieving historical correction parameters corresponding to the maximum overall overlap from the operation log information, and obtaining correction accuracy of the sampling arm after applying various historical correction parameters;

S64, constructing a size ranking table, and inputting the correction accuracy of the sampling arm after applying various historical correction parameters into the size ranking table for sorting, to obtain maximum correction accuracy;

S65, obtaining the historical correction parameters corresponding to the maximum correction accuracy, and delivering historical correction parameters corresponding to the maximum correction accuracy to a controller of the in vitro trans-dermal diffusion apparatus for gas exposure as the optimal correction parameter, and the controller is configured to correct the sampling arm based on the optimal correction parameter.

The calculation principle for the overall overlap between the real-time posture 3D model and the abnormal posture 3D model corresponding to various posture anomalies of the sampling arm is the same as the calculation principle for the overall overlap between the real-time posture 3D model and the preset posture 3D model, and will not be repeated here.

It should be noted that the operation log information of the in vitro transdermal diffusion apparatus for gas exposure refers to a series of data and information automatically recorded by the system during the experiment. These include the operational status of the apparatus, the process of operation, test parameters, and any abnormal conditions. This information is critical for monitoring the test process, analyzing the test results, optimizing experimental methods, and improving equipment design.

The step of performing a reduce redundancy process to the real-time posture image to obtain a reduced redundancy real-time posture image includes the following steps.

Performing a gray value processing to the real-time posture image to obtain the grayscale posture image.

Performing a feature extraction on the grayscale posture image to obtain feature descriptors of the grayscale posture image.

Calculating a similarity between the feature descriptors based on KD-tree algorithm, and calculating a real-time similarity matrix for the posture image based on the similarity between the feature descriptors. The matrix elements represent the similarity between the feature descriptors in the grayscale posture image.

Obtaining a pre-established standard similarity matrix, and calculating a difference between the actual similarity in the real-time similarity matrix and the standard similarity at the same positions to get several similarity differences.

Marking the image regions in the grayscale posture image where the similarity difference exceeds the preset threshold as redundant image areas. The pixel values at each position in the redundant image areas are obtained and used to form a data matrix.

Calculating covariance between each pixel position in the redundant image area, and constructing a covariance matrix based on the covariance. Applying an eigenvalue decomposition to the covariance matrix to obtain eigenvalues and their corresponding eigenvectors.

Selecting top k largest eigenvalues and their corresponding eigenvectors to obtain selected principal components. Projecting the data matrix is onto the selected principal components to obtain a reduced-dimension data matrix.

Converting the reduced-dimension data matrix into image format to obtain the reduced-redundancy redundant image area. The real-time posture image is updated based on the reduced-redundancy redundant image area, resulting in the reduced-redundancy real-time posture image.

It should be noted that real-time posture images may be affected by image noise, lighting changes, occlusions, and other factors, leading to redundancy in the image. Therefore, after obtaining the real-time similarity matrix for the posture image, the comparison and analysis of the real-time similarity matrix with the standard similarity matrix are performed to identify redundant image areas. After identifying the redundant image areas, principal component analysis (PCA) is applied to reduce redundancy in these areas, resulting in a reduced-redundancy real-time posture image. By analyzing the similarity matrix, redundant image areas are identified, and PCA is used to reduce redundancy in these areas, reducing redundant information in the image. This helps reduce the data size of the image, improve image quality, and enhance the performance and efficiency of image processing tasks, making it more accurate in determining whether the sampling arm needs to be corrected.

The step of constructing a real-time posture 3D model of the sampling arm at the preset time point based on the reduced redundancy real-time posture image includes the following steps.

Extracting feature points from the reduced-redundancy real-time posture image based on oriented fast and rotated brief (ORB) algorithm, to obtain several feature points.

Selecting a random feature point as a coordinate origin, and building a 3D coordinate system based on the coordinate origin. The 3D coordinates of all the feature points are obtained in the 3D coordinate system, and a feature point coordinate set is generated based on the 3D coordinates.

Importing the feature point coordinate set into 3D modeling software for model construction, to obtain an initial real-time posture 3D model.

Performing a feature decomposition on the initial real-time posture 3D model to obtain an orthogonal matrix and a diagonal matrix. The diagonal matrix and orthogonal matrix are multiplied to obtain a transformation matrix, from which the dual vectors are extracted. These dual vectors are used to construct the world coordinate system.

Importing the orthogonal matrix and the diagonal matrix into the world coordinate system, to obtain a world coordinate set.

Obtaining a limit coordinate point set from the world coordinate set, and retrieving the vertex coordinate points in the initial real-time posture 3D model to obtain the vertex coordinate point set.

Comparing the limit coordinate points and the vertex coordinate points at the same positions, and defining the limit coordinate points and vertex coordinate points that have the same coordinates as normal points, while defining those with different coordinates as singular points.

Obtaining the singular point positions in the initial real-time posture 3D model, and these positions are corrected based on the corresponding limit coordinate points, resulting in the real-time posture 3D model.

It should be noted that when generating the initial real-time posture 3D model based on the feature point coordinate set, some regions of the model may contain minor defects such as cracks or protrusions. To improve the model quality, the method automatically analyzes and identifies the singular points, which are the minor defects, and then repairs these singular points, resulting in a model with higher fidelity. This enables a more accurate determination of whether the sampling arm requires correction.

The control method for an in vitro trans-dermal diffusion apparatus for gas exposure is processed by a control equipment for an in vitro trans-dermal diffusion apparatus for gas exposure.

As shown in FIG. 10, the control equipment for the in vitro trans-dermal diffusion apparatus for gas exposure includes: a processor 1001 (such as Central Processing Unit, CPU), a communication bus 1002, an input port 1003, an output port 1004, and a memory 1005. Among them, the communication bus 1002 is used to achieve connection communication between these components; the input port 1003 is used for data input; and the output port 1004 is used for data output, and the memory 1005 can be high-speed RAM memory or non volatile memory, such as disk memory, non-transitory computer-readable storage medium. Optionally, memory 1005 is a storage device independent of the aforementioned processor 1001.

The memory 1005, as a non-volatile readable storage medium, may include an operating system, network communication module, application program module, and a program for controlling the in vitro trans-dermal diffusion apparatus for gas exposure. The network communication module is mainly used to connect to servers and communicate data with them; And processor 1001 is used to call the program to process the method stored in memory 1005, and execute all steps of the control method for the in vitro trans-dermal diffusion apparatus for gas exposure mentioned above.

The above are only some embodiments of the present disclosure, and neither the words nor the drawings can limit the protection scope of the present disclosure. Any equivalent structural transformation made by using the contents of the specification and the drawings of the present disclosure under the overall concept of the present disclosure, or directly/indirectly applied in other related technical fields are included in the protection scope of the present disclosure.

What is claimed is:

1. An in vitro trans-dermal diffusion apparatus for gas exposure, wherein, comprising a gas generator, an exposure chamber, an exhaust treating device, a sampling arm, a diffusion chamber, a liquid replenishment tank, and a sample bottle rack;
wherein the diffusion chamber comprises a skin cover, a receptor pool, gaskets, a skin membrane, a gas collecting port, a sampling port, and a liquid replenishment port;
wherein the skin membrane is clamped between the skin cover and the receptor pool, and each gasket is an elliptical ring; and a gas collecting port is upwardly inclined at the receptor pool, and the gas collecting port is at a position of 1 to 2 mm below a top of the receptor pool, with the sampling port and the replenishment port positioned opposite each other at a bottom of the diffusion chamber; and
the gas generator is configured to generate a target gas with constant humidity, and the target gas is configured to continuously enter the exposure chamber through a peristaltic pump at a constant speed via a heating tube; and
the exposure chamber is a temperature-controlled box with gas inlet holes and outlet holes on both sides, the gas inlet holes and the outlet holes are connected to exhaust hoses; and
mounting holes of the diffusion chamber are evenly distributed at the bottom of the exposure chamber; and
the replenishment tank is connected to the peristaltic pump to deliver liquid to the replenishment port; and
the exposure chamber is equipped with an air inlet and an air outlet; and the exhaust treating device is located on a side of the outlet and communicated with the air outlet.

2. The in vitro trans-dermal diffusion apparatus for gas exposure according to claim 1, wherein the sampling arm comprises a supporting column, a sliding rail, a first sampling arm, a second sampling arm, and a sampling needle, wherein the sliding rail is positioned at a top of the supporting column, and the first sampling arm is configured to move along the sliding rail via an X-axis stepper motor.

3. The in vitro trans-dermal diffusion apparatus for gas exposure according to claim 2, wherein the second sampling arm comprises a rotating joint and a telescopic rod, with a sampling needle holder connected to an end of the telescopic rod.

4. The in vitro trans-dermal diffusion apparatus for gas exposure according to claim 1, wherein a top structure of the receptor pool is inclined elliptical hollow with a protrusion at an upper end; a top structure of the skin cover is inclined elliptical hollow, and a bottom of the skin cover is defined with a groove that matches the protrusion of the receptor pool.

5. The in vitro trans-dermal diffusion apparatus for gas exposure according to claim 1, wherein the sample bottle rack is configured to hold sample bottles and wash solution bottles.

6. The in vitro trans-dermal diffusion apparatus for gas exposure according to claim 1, wherein the in vitro trans-dermal diffusion apparatus for gas exposure further comprises a camera.

7. A control method for an in vitro trans-dermal diffusion apparatus for gas exposure, applied to the in vitro trans-dermal diffusion apparatus for gas exposure of claim 1, wherein comprising the following steps:
when the in vitro trans-dermal diffusion apparatus for gas exposure is on a working state, capturing a real-time posture image of a sampling arm at a preset time point through a camera, and performing a reduce redundancy process to the real-time posture image to obtain a reduced redundancy real-time posture image; and
constructing a real-time posture three-dimensional (3D) model of the sampling arm at the preset time point based on the reduced redundancy real-time posture image, and obtaining a preset posture 3D model of the sampling arm at the preset time point; and
calculating an overall overlap between the real-time posture 3D model of the sampling arm and the preset posture 3D model at a same preset time point through a model attribute map method; and
comparing the overall overlap with a preset threshold; and
if the overall overlap between the real-time posture 3D model and the preset posture 3D model is greater than the preset threshold, which indicates that the posture of the sampling arm is normal, and no correction is applied to the sampling arm; and
if the overall overlap is not greater than the preset threshold, which indicates that the posture of the sampling arm is abnormal posture, and an optimal correction parameter is generated to correct the sampling arm based on the optimal correction parameter.

8. The control method for the in vitro trans-dermal diffusion apparatus for gas exposure according to claim 7, wherein the step of calculating an overall overlap between the real-time posture 3D model of the sampling arm and the preset posture 3D model at a same preset time point through a model attribute map method comprises the following steps:
constructing a virtual space, and importing the real-time posture 3D model and the preset posture 3D model into the virtual space, to obtain a real-time posture assembly reference plane of the real-time posture 3D model and a preset posture assembly reference plane of the preset posture 3D model; and
in the virtual space, aligning the real-time posture assembly reference plane and the preset posture assembly reference plane, and merging the real-time posture assembly reference plane and the preset posture assembly reference plane to obtain a merged posture 3D model; and
dividing the merged posture 3D model into several sub-regions, and each sub-region represents as a polygon; and
for each sub-region, calculating a ratio of the overlap volume between the real-time posture 3D model and the preset posture 3D model to a total volume of the sub-region through Boolean operations, to obtain an overlap degree between the real-time posture 3D model and the preset posture 3D model in each sub-region; and
aggregating overlap degrees of the sub-regions through a weighted sum method to calculate the overall overlap between the real-time posture 3D model and the preset posture 3D model.

9. The control method for an in vitro trans-dermal diffusion apparatus for gas exposure according to claim 7, wherein the step of if the overall overlap is not greater than the preset threshold, which indicates that the posture of the sampling arm is abnormal posture, and an optimal correction parameter is generated to correct the sampling arm based on the optimal correction parameter comprises the following steps:
obtaining operation log information of the in vitro trans-dermal diffusion apparatus for gas exposure, and extracting abnormal posture 3D models corresponding to various abnormal postures of the sampling arm from the operation log; and
if the overall overlap between the real-time posture 3D model and the preset posture 3D model is not greater than the preset threshold, obtaining the real-time posture 3D model of the sampling arm is obtained, and calculating the overall overlap between the real-time posture 3D model and the abnormal posture 3D models corresponding to abnormal postures of the sampling arm;
selecting the abnormal posture 3D model corresponding to the maximum overall overlap, and retrieving historical correction parameters corresponding to the maximum overall overlap from the operation log information, and obtaining correction accuracy of the sampling arm after applying various historical correction parameters;
constructing a size ranking table, and inputting the correction accuracy of the sampling arm after applying various historical correction parameters into the size ranking table for sorting, to obtain maximum correction accuracy;
obtaining the historical correction parameters corresponding to the maximum correction accuracy, and delivering historical correction parameters corresponding to the maximum correction accuracy to a controller of the in vitro trans-dermal diffusion apparatus for gas exposure as the optimal correction parameter, and the controller is configured to correct the sampling arm based on the optimal correction parameter.
